(19) United States
(12) Patent Application Publication
(10) Pub. No.: US 2012/0197119 A1
(43) Pub. Date: Aug. 2, 2012

(54) Treatment instrument

(75) Inventor: Hideo Takachi, Yokohama-shi (JP)

(73) Assignee: OLYMPUS MEDICAL SYSTEMS CORP., Tokyo (JP)

(21) Appl. No.: 13/016,067

(22) Filed: Jan. 28, 2011

(51) Int. Cl. A61B 8/12

(52) U.S. Cl. 600/439

(57) ABSTRACT

A treatment instrument that is inserted in a freely advancing and retracting manner into the channel of an ultrasonic endoscope, includes a treatment member that has a distal end and a proximal end and an outer surface that is a circular cylindrical shape in which a plurality of dimples are formed indenting into the outer surface. The length component that is measured in the direction of the center axis of the treatment member and which is the distance between the rear edge, which is positioned closest to the proximal end, and the deepest part of the dimple is shorter than the length component that is measured in the direction of the center axis and which is the distance between the front edge which is closest to the distal end and the deepest part.

### BACKGROUND OF THE INVENTION

1. Field of the Invention

The present invention relates to a treatment instrument.

2. Description of Related Art

The use of ultrasound to visualize a treatment instrument positioned in an area that cannot be viewed optically is a conventionally known technique. When using ultrasound to visualize a treatment instrument, a probe is typically employed that both generates the ultrasound waves and detects the ultrasound waves that are reflected by the outer surface of the treatment instrument. An image of the treatment instrument is then obtained by generating an ultrasound image based on the ultrasound waves that are detected by the probe.

Examples of treatment instruments that enable visualization using ultrasound images include, for example, the treatment instrument (puncture needle) disclosed in Japanese Patent Application, Publication No. 2006-101915 in which a roughly hemispherical concave groove is formed to the outer surface of the treatment member (outer barrel needle). Because the concave grooves are hemispherical in shape in the treatment instrument disclosed in Japanese Patent Application, Publication No. 2006-101915, it is possible to obtain ultrasound waves that reflect toward the probe even when the positional relationship between the treatment member and the probe does not coincide.

Further, the pamphlet for International Patent Application, Publication No. WO 07/013130 discloses forming a plurality of corner cube mirrors to the outer surface of the treatment member with the goal of making the image of the treatment member (needle barrel main body) in the ultrasound image clearer.

In addition, ultrasonic endoscopy is widely employed in which a probe is provided to the distal end of the inserted part of the endoscope that is introduced into the body, so that a procedure can be carried out while observing the position of the treatment instrument using the ultrasound image. Examples of a treatment instrument employed with this type of ultrasonic endoscope include the treatment instrument (ultrasonic puncture needle) disclosed in Japanese Patent Application, Publication No. 2003-190179 which is provided with a treatment member (needle barrel) in which a plurality of annualar grooves are formed to the outer surface thereof.

### SUMMARY OF THE INVENTION

The first aspect of the present invention relates to a treatment instrument that can be inserted in a freely advancing and retracting manner into the channel of an ultrasonic endoscope, and which has a treatment member that has a distal end and a proximal end and an outer surface that is a circular cylindrical shape in which a plurality of dimples are formed indenting into the outer surface, this treatment instrument being characterized in that the length component which is measured in the direction of the center axis of the treatment member and which is the distance between the rear edge, which is positioned closest to the proximal end, and the deepest part of the dimple, is shorter than the length component which is measured in the direction of the center axis and which is the distance between the front edge which is closest to the distal end and the deepest part.

### BRIEF DESCRIPTION OF THE DRAWINGS

FIG. 1 is a perspective view showing the approximate structure of the treatment instrument and ultrasonic endoscope according to a first embodiment of the present invention.

FIG. 2 is a side view showing a cross-section through part of this same treatment instrument.

FIG. 3 is a cross-sectional view showing the structure at the distal end side of the inserted body in the same treatment instrument.

FIG. 4 is a partial cross-sectional view showing an enlarged view of the distal end portion of the needle barrel in the same treatment instrument.

FIG. 5 is a view showing an enlargement of the part in FIG. 4.

FIG. 6 is a cross-sectional view along the line A-A in FIG. 2.

FIG. 7 is a cross-sectional view along the line B-B in FIG. 2.

FIG. 8 is a half sectional view showing the vicinity of the sheath adjuster in the operation section of the same treatment instrument.

FIG. 9 is a half sectional view showing an enlargement of the needle slider in the same operation section.

FIG. 10 is a view showing an enlargement of the same needle slider, as seen along the arrow C shown in FIG. 2.

FIG. 11A is a side view showing the stylette in the same treatment instrument.

FIG. 11B is a side view showing an example of another structure of the stylette.

FIG. 12 is a cross-sectional view along the line D-D in FIG. 11A

FIG. 13 is a perspective view showing the arrangement when the treatment instrument is stored inside the storage bag for holding the same treatment instrument.

FIG. 14 is a planar view of the tray in the same storage bag.

FIG. 15 is a rear view showing the arrangement when the same treatment instrument is attached to the same tray.

FIGS. 16 though 18 are explanatory views for explaining the sequence for removing the same treatment instrument from the same storage bag.

FIGS. 19 though 22 are views for explaining the action when using the same treatment instrument.

FIG. 23 is a schematic view showing the reflected state of the ultrasound waves when the needle barrel of the same treatment instrument has punctured the tissue.

FIG. 24 is a schematic view showing the reflected state of the ultrasound waves in a needle barrel having the conventional dimples.

FIG. 25 is a schematic view showing the process of a treatment using the same treatment instrument.

FIG. 26A and FIG. 26B are schematic views showing a modification of the same treatment instrument.

FIG. 27A and FIG. 27B are schematic views showing another modification of the same treatment instrument.

FIG. 28A and FIG. 28B are schematic views showing another modification of the same treatment instrument.

FIG. 29 is a view showing another modification of the storage bag for storing the same treatment instrument, and is a planar view of the tray.

FIG. 30 is a side view of the tray in another modification.

### DETAILED DESCRIPTION OF THE INVENTION

The treatment instrument according to a first embodiment of the present invention will now be explained.

The treatment instrument according to this embodiment is a medical device that can be inserted in a freely advancing and retracting manner into the channel of an ultrasonic endoscope, and is employed together with the ultrasonic endoscope to perform treatments within the body.

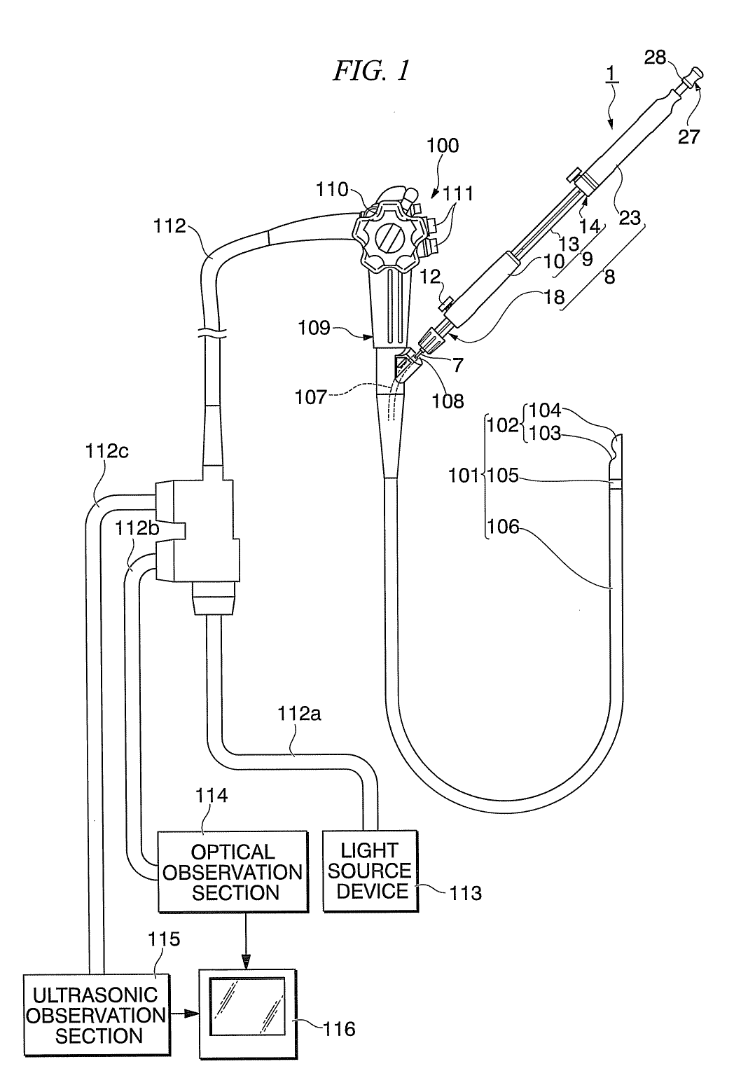

An overview of the structure of the ultrasonic endoscope 100 which is used together with the treatment instrument 1 will first be explained with reference to FIG. 1. FIG. 1 is a perspective view showing the overview structure of the treatment instrument and ultrasonic endoscope according to this embodiment.

As shown in FIG. 1, the ultrasonic endoscope 100 is provided with an inserted part 101 which is inserted into the body from its distal end; an operation section 109 which is attached to the proximal end of the inserted part 101; a universal cord 112, one end of which is connected to the side of the operation section 109; a light source device 113 which is connected to the other end of the universal cord 112 via a branch cable 112a; an optical observation section 114 which is connected to this same other end of the universal cord 112 via a branch cable 112b; and an ultrasonic observation section 115 which is connected to this same other end of the universal cable 112 via a branch cable 112c.

The inserted part 101 is provided from its distal end side with, in sequence, a distal end rigid part 102, a curved part 105 and a flexible tubular part 106.

The distal end rigid part 102 is provided with an optical image capture mechanism 103 for carrying out optical observation, and an ultrasonic scanning mechanism 104 for carrying out ultrasound observation.

The optical image capture mechanism 103 is provided with various structures, not shown in the figures, such as an image capture optical system in which the field of view is to the front of the distal end rigid part 102, image sensors such as CCD, CMOS and the like for detecting the image of the object which incident via the image capture optical system, and a CCU for controlling the operations of the image sensors.

The ultrasonic scanning mechanism 104 is provided with an ultrasonic vibrating element for generating ultrasound waves and an ultrasonic receiver for receiving ultrasound waves (neither of which are shown in the figures). The ultrasonic scanning mechanism 104 is designed such that the ultrasound waves generated by the ultrasonic vibrating element hit the target object to be visualized and are reflected, and these reflected waves are received at the ultrasonic receiver. The ultrasonic receiver then outputs a signal based on these received ultrasound waves to the ultrasonic observation section 115.

The curved part 105 is formed to be cylindrical in shape, and curves in specific directions such as up, down, left, and right, by means of a pulling manipulation of angle wires at operation section 109. These angle wires are not shown in the figures but are fixed to the distal end of the curved part 105 and extend to the operation section 109.

The flexible tubular part 106 is a cylindrical member that is formed to be soft so as to be able to guide the distal end rigid part 102 to a specific position within the digestive tract or a body cavity.

A cylindrical channel 107, for insertion of the treatment instrument 1, and a tubular path, not shown in the figures for relaying and suctioning air and water, are provided within the curved part 105 and the flexible tubular part 106 respectively.

One end of the channel 107 opens at the distal end of the distal end rigid part 102, and the other end of the channel 107 opens on the lateral surface of the distal end side of the operation section 109. A distal end mouthpiece 108 which is formed in the shape of a flange is fixed in place to the other end of the channel 107. The treatment instrument 1, which is used together with the ultrasonic endoscope 100, may be fixed in place to the distal end mouthpiece 108.

The operation section 109 has an outer surface that is formed to enable it to be held in the hand of the user operating the ultrasonic endoscope 100. The operation section 109 is provided with a bending manipulation mechanism 110 for bending the curved part 105 by applying traction on an angle wire, and a plurality of switches 111 for sending or suctioning gas or water via the tubular path.

The light source device 113 is for generating the illumination needed for image captured by the optical image capture mechanism 103.

The optical observation section 114 is designed to reflect the image picked up by the image sensor of the optical image capture mechanism 103 onto a monitor 116.

The ultrasonic observation section 115 receives the signal output from the ultrasonic scanning mechanism 104, generates an image based on this signal and reflects this onto the monitor 116.

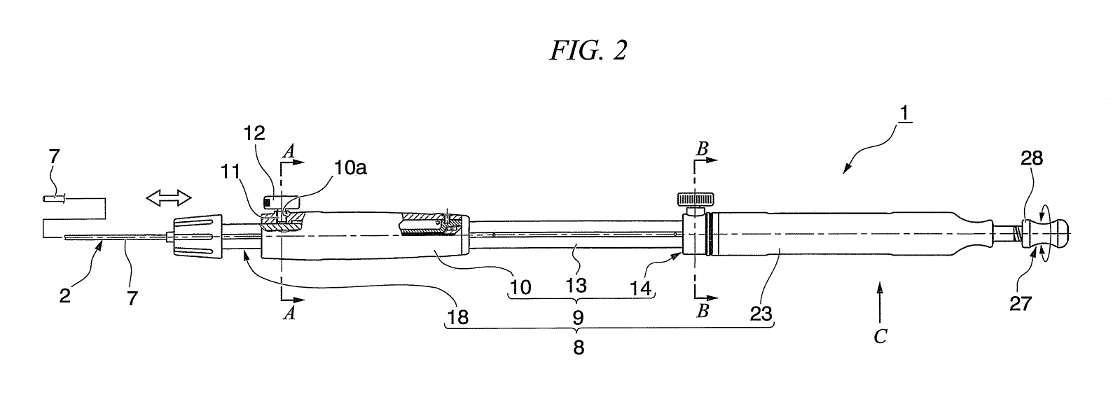

Next, the configuration of the treatment instrument 1 of this embodiment which is used together with the ultrasonic endoscope 100 will be explained with reference to FIGS. 2 through 12.

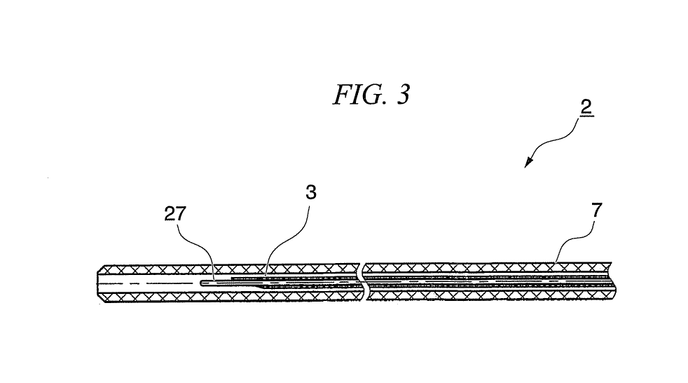

FIG. 2 is a side view showing the treatment instrument 1 as seen in partial cross-section. FIG. 3 is a view showing the structure of the distal end side of the inserted body 2 and is a cross-sectional view along the axial direction of the inserted body 2.

As shown in FIG. 2, the treatment instrument 1 is provided with an inserted body 2, an operation section 8 and a stylette 27.

As shown in FIG. 3, the inserted body 2 is provided with a needle barrel 3 (treatment member) that is cylindrical in shape and has a distal end and a proximal end, and a cylindrical sheath 7 into which the needle barrel 3 is inserted.

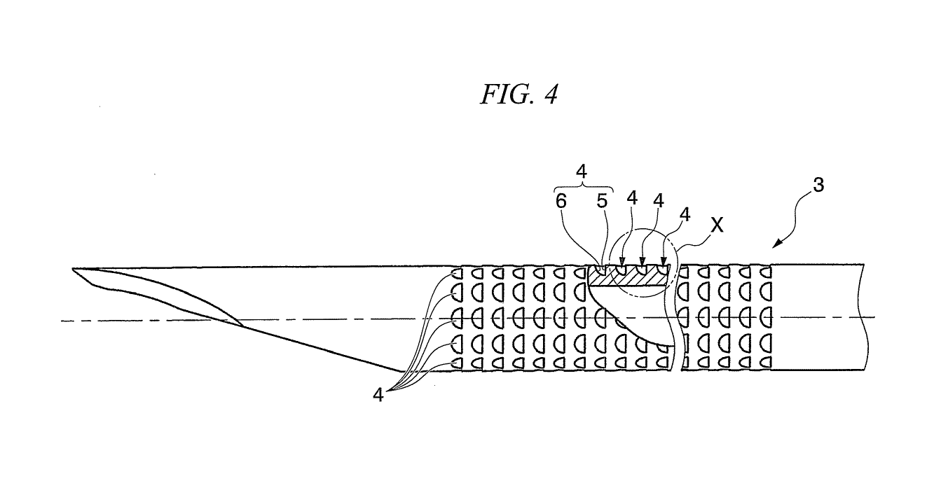

FIG. 4 is a partial cross-sectional view showing an enlargement of the distal end portion of the needle barrel 3.

As shown in FIG. 4, the distal end of the needle barrel 3 is cut diagonally so that it is sufficiently sharp to enable piercing of a biological tissue. A plurality of dimples 4 are provided on the outer surface of the needle barrel 3 in the form of radially inwardly directed indentations in the outer surface of the needle barrel 3.

It is preferable that the material for the needle barrel 3 has both flexibility and elasticity to easily return to a linear form even when bent by an external force. For example, an alloy material such as a stainless alloy, or nickel titanium alloy may be used as the material for needle barrel 3.

Dimples 4 are formed in rows around the circumferential direction of the outer surface of the needle barrel 3 with a slight space open between adjacent dimples. The dimples 4 are also formed in rows along the center axis of the needle barrel 3 with a slight space open between adjacent dimples.

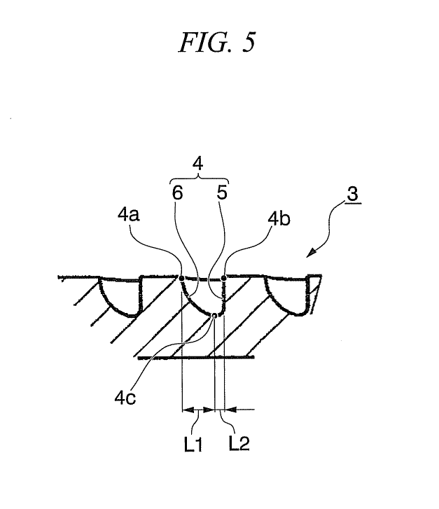

FIG. 5 is an expanded view of the component indicated by the symbol X in FIG. 4.

As shown in FIG. 5, the inner surface of the dimples 4 is formed of a planar surface part 5 which is parallel to a plane intersecting with the center axis of the needle barrel 3, and a concave surface part 6 consisting of a concave surface that is formed closer to the distal end side of the needle barrel 3 than the planar surface part 5.

In this embodiment, the concave surface part 6 forms part of a spherical surface. Specifically, the inner surface of the dimples 4 in this embodiment is shaped to lie along the profile of the outer surface of one quarter of a sphere formed when a sphere is sectioned by two planes that intersect through the center of the sphere.

The length component L2 which is measured along the center axis of the needle barrel 3 and is the distance between the rear edge 4b, which is positioned closer to the proximal end of the needle barrel 3, and the deepest part 4c, which is at the most radially inward side, is shorter than the length component L1 which is measured along the center axis and is the distance between the front edge 4a, which is closest to the distal end, and the deepest part 4c of the dimples 4.

Note that the length component L2 is exaggerated in FIG. 5 to facilitate visualization. However, in this embodiment, the length component L2, which is measured along the center axis of the needle barrel 3 and is the distance between the rear edge 4b and the deepest part 4c, is approximately 0.

As shown in FIG. 2, sheath 7 consists of a flexible tubular member such as metallic coil or resin, and extends from the distal end of the operation section 8. Examples of resins applicable for the material of the sheath 7 include polyetheretherketone (PEEK), fluorine resins, olefin resins, urethane resins, and nylon (polyamide type) resins, among others. Note that the proximal end of the sheath 7 is fixed in place to the operation section main body 9, explained below, within the operation section 8.

The operation section 8 is provided with an operation section main body 9, a sheath adjuster 18 provided to the distal end side of the operation section main body 9, and a needle slider 23 which is provided to the proximal end side of the operation section main body 9.

The operation section main body 9 is provided with a grip 10 held by the user during use of the treatment instrument 1, a slide rail 13 which extends from the grip 10 toward the proximal end side of the operation section 8, and a slider stopper 14 which is provided to the slide rail 13.

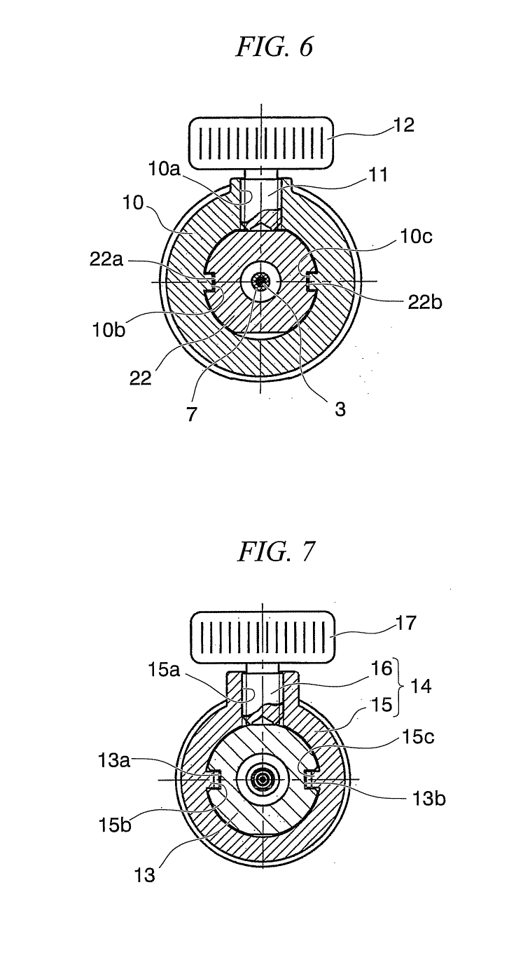

FIG. 6 is a cross-sectional view along the line A-A in FIG. 2.

As shown in FIGS. 2 and 6, the grip 10 is formed to be roughly cylindrical. A screw hole 10a penetrating in the width direction of the grip 10 is formed to the outer surface of grip 10 for attaching a screw 11 for fixing the sheath adjuster 18 in place. In addition, a pair of projections 10b,10c are formed to the inside of the grip 10 and engage in the groove of the slide rail 22, explained below, of the sheath adjuster 18.

The distal end of the screw 11 can contact with the outer surface of the slide rail 22 described below. A large diameter part 12 that has a diameter larger than the shaft is provided to the head part of screw 11. In addition, a plurality of grooves is formed to the outer periphery of the large diameter part 12. As a result, the screw 11 can easily be turned with the hand.

FIG. 7 is a cross-sectional view along the line B-B in FIG. 2.

As shown in FIG. 2 and FIG. 7, the slide rail 13 is a roughly cylindrical member for advancing and retracting manipulation of the needle slider 23 along the center axis. Grooves 13a,13b are formed to the outer surface of the needle slider 23 and extend parallel to the center axis. The grooves 13a and 13b are disposed to be opposite one another in the radial direction of the slide rail 13. A portion of the outer peripheral surface of the slide rail 13 which comes into contact with the distal end of the screw 16 is formed to be flat and will be explained further below.

The slider stopper 14 has an annular member 15, into which the slide rail 13 is inserted and which has a screw hole 15a formed penetrating in the width direction thereof, and a screw 16 which screws into the screw hole 15a of the annular member 15.

The annular member 15 has an inner diameter that is slightly larger than the diameter of the slide rail 13, and is attached to the slide rail 13 in a manner to enable advancing and retracting operation along the center axis of the slide rail 13. Projections 15b,15c are formed to the annular member 15 that are inserted into the grooves 13a,13b. The annular member 15 does not rotate in the circumferential direction of the slide rail 13.

The distal end of the screw 16 is designed to enable contact with the outer peripheral surface of the slide rail 13. The annular member 15 can be fixed in place to the slide rail 13 by screwing the screw 16 into the screw hole 15a of the annular member 15. Further, a large diameter part 17, which is formed to have a large diameter, is provided to the proximal end of the screw 16. A plurality of grooves is formed to the outer periphery of the large diameter part 17. As a result, the screw 16 can be easily turned with the hand.

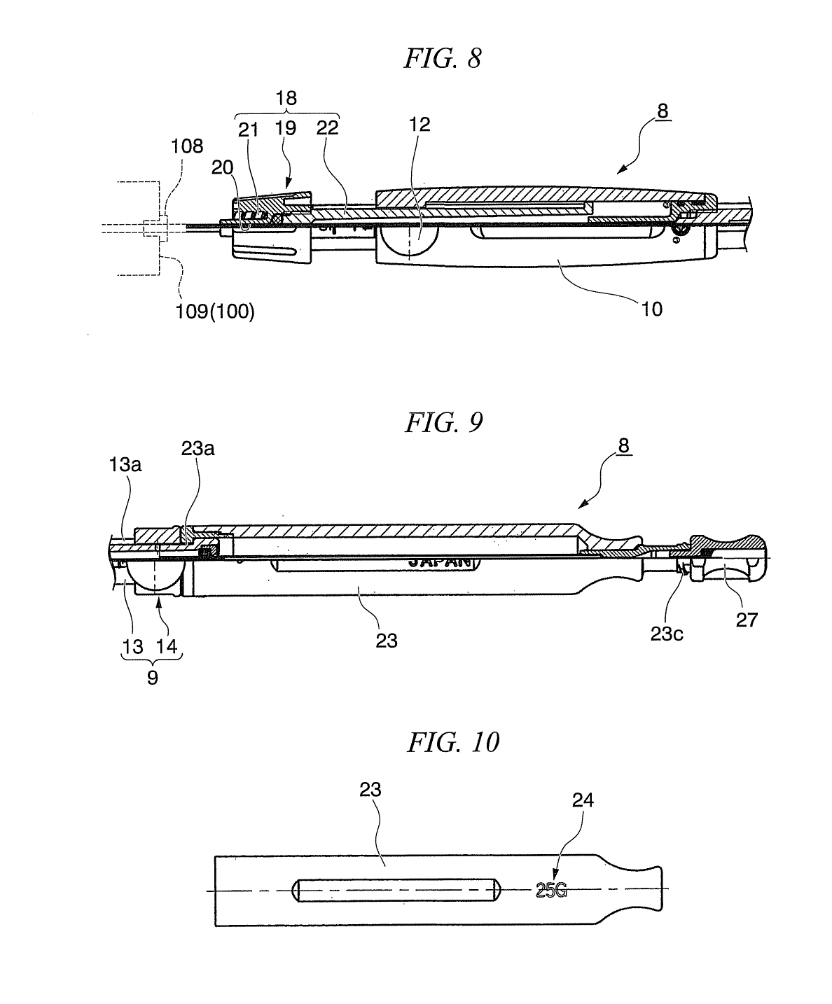

FIG. 8 is a half sectional view showing the vicinity of the sheath adjuster 18 in the operation section 8.

The sheath adjuster 18 is for adjusting the amount that the sheath 7 is projected out from the distal end of the channel 107 of the ultrasonic endoscope 100 as shown in FIG. 1. As shown in FIG. 8, the sheath adjuster 18 is provided with a fixing screw 19 for fixing the base mouthpiece 108 of the ultrasonic endoscope 100 and a slide rail 22 which is fixed to the fixing screw 19 and is inserted inside the grip 10.

A through hole 20 into which the sheath 7 is inserted and a screw groove 21 for attaching to the distal end mouthpiece 108 are formed to the fixing screw 19. In addition, convexities and concavities are formed to the outer surface of the fixing screw 19 for stopping sliding when attaching and detaching the fixing screw 19 to and from the distal end mouthpiece 108.

As shown in FIGS. 6 and 8, the slide rail 22 is a cylindrical member in which a pair of grooves are formed extending parallel to the center axis. The sheath 7 and the needle barrel 3 are inserted within the slide rail 22. The distal end of the slide rail 22 is fixed in place by the fixing screw 19. The slide rail 22 is inserted into the grip 10. A pair of grooves 22a,22b are formed to the outer peripheral surface of the slide rail 22, these grooves 22a,22b elongating in the axial direction and being formed opposite one another about the radial direction of the slide rail 22. Projections 10b,10c formed to the grip 10 engage in these grooves 22a,22b respectively.

The distal end of the screw 11 can come into contact with the outer surface of the slide rail 22 inside the grip 10 when the screw 11 is screwed into the screw hole 10a of the grip 10. As a result, when the screw 11 is screwed into the grip 10, the slide rail 22 and the grip 10 are fixed in place due to the distal end of the screw 11 pressing against the outer surface of the slide rail 22. In addition, the grip 10 and the slide rail 22 of the sheath adjuster 18 can move relative to one another along the longitudinal direction of the groove when the screw 11 is loosened.

FIG. 9 is a half sectional view showing an enlargement of the needle slider 23 in the operation section 8. FIG. 10 is a view showing an enlargement of the needle slider 23 in the operation section 8 and is viewed in the direction of arrow C in FIG. 2.

As shown in FIG. 9, the needle slider 23 is a cylindrical member into which the slide rail 13 of the operation section main body 9 is inserted. A pair of projections 23a (only one of which is shown in the figures) that engage in the grooves 13a,13b of the slide rail 13 are formed to the needle slider 23. The distal end of the needle slider 23 comes into contact with the proximal end of the slider stopper 14. A screw thread 23c for screwing a knob 28 and a syringe 120 explained below, is formed to the proximal end of the needle slider 23.

The outer surface of the needle slider 23 has a circular cylindrically shaped outer surface to enable gripping by the user who is using the treatment instrument 1. In addition, as shown in FIG. 10, a gauge number display 24 is formed to the outer surface of the needle slider 23 which has engraved, etc. the gauge number which indicates the diameter of the needle barrel 3.

While not shown in detail, the proximal end of the needle barrel 3 is fixed to the inside of the needle slider 23. A stylette 27, explained further below, can be inserted from the proximal end of the needle barrel 3 which is fixed to the needle slider 23. The needle slider 23 can be moved along the center axis in the grooves 13a,13b (see FIG. 7) of the slide rail 13, to enable projection and retraction of the needle barrel 3 from the sheath 7.

The needle slider 23 and the slide rail 13 are connected so that the needle slider 23 and the slide rail 13 do not disconnect when moving the needle slider 23 toward the proximal end side of the slide rail 13. Once the needle slider 23 has been completely moved to the proximal end side of the slide rail 13, the distal end of the needle barrel 3, which is fixed to the needle slider 23, is pulled into the distal end of the sheath 7 and has a positional relationship in which it is covered by the sheath 7.

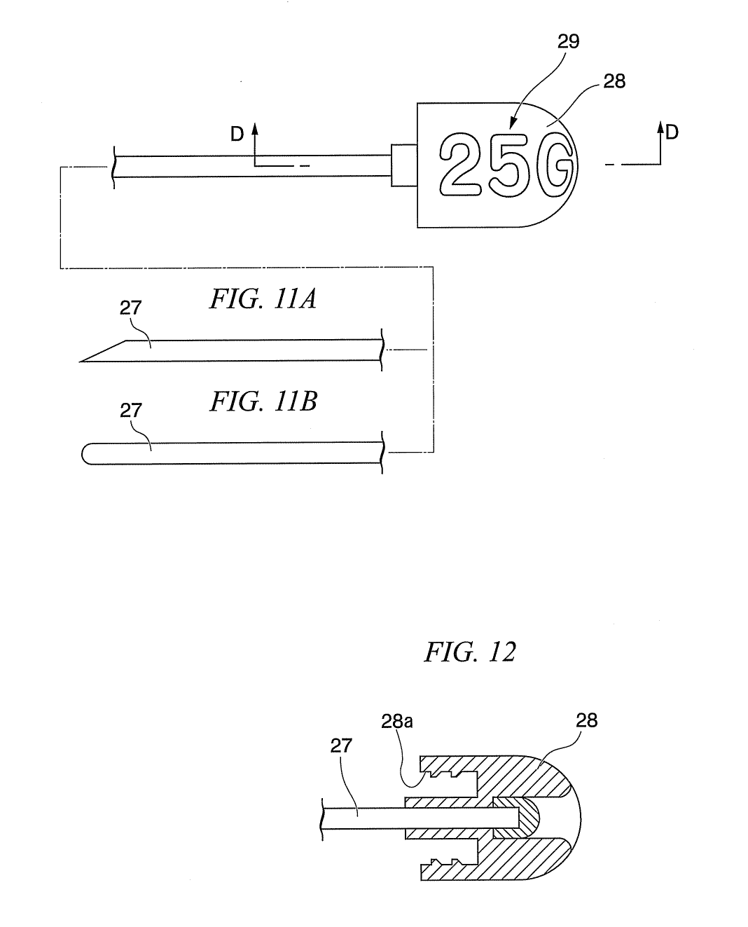

FIG. 11A is a side view of the stylette 27. FIG. 12 is a cross-sectional view along the line D-D in FIG. 11A.

As shown in FIG. 11A and FIG. 12, the stylette 27 is a metallic linear member having a diameter that enables insertion in a freely advancing and retracting manner inside the needle barrel 3. The distal end of the stylette 27 is formed to be sharp, and a knob 28, which is formed of resin or the like, is provided to the proximal end of the stylette 27. Note that as shown in FIG. 11B, the distal end of the stylette 27 may be formed to be roughly semicircular in shape.

A gauge number indicator 29 is formed to the knob 28 that is provided to the proximal end of the stylette 27. This gauge number indicator 29 displays in engraving or the like the gauge number of the needle barrel 3 into which stylette 27 can be suitably introduced. In other words, provided that the stylette 27 has a gauge number displayed on the knob 28 that is the same value as the gauge number that is displayed on the gauge display 24, then this stylette 27 can be used by suitably inserted into the needle barrel 3. As a result, it is possible to avoid mistakes in a procedure when using a plurality of treatment instruments 1 having different needle barrel 3 and the stylette 27 diameters.

As shown in FIG. 12, a groove 28a is formed to the knob 28 for engaging with the screw tread 23c (see FIG. 9) formed to the proximal end of the needle slider 23. By aligning the screw groove 28a of the knob 28 in the screw thread 23c of the needle slider 23, the stylette 27 can be fixed in place to the needle slider 23. The distal end of the stylette 27 is designed to be positioned projecting out slightly from the distal end of the needle barrel 3 (see FIG. 3).

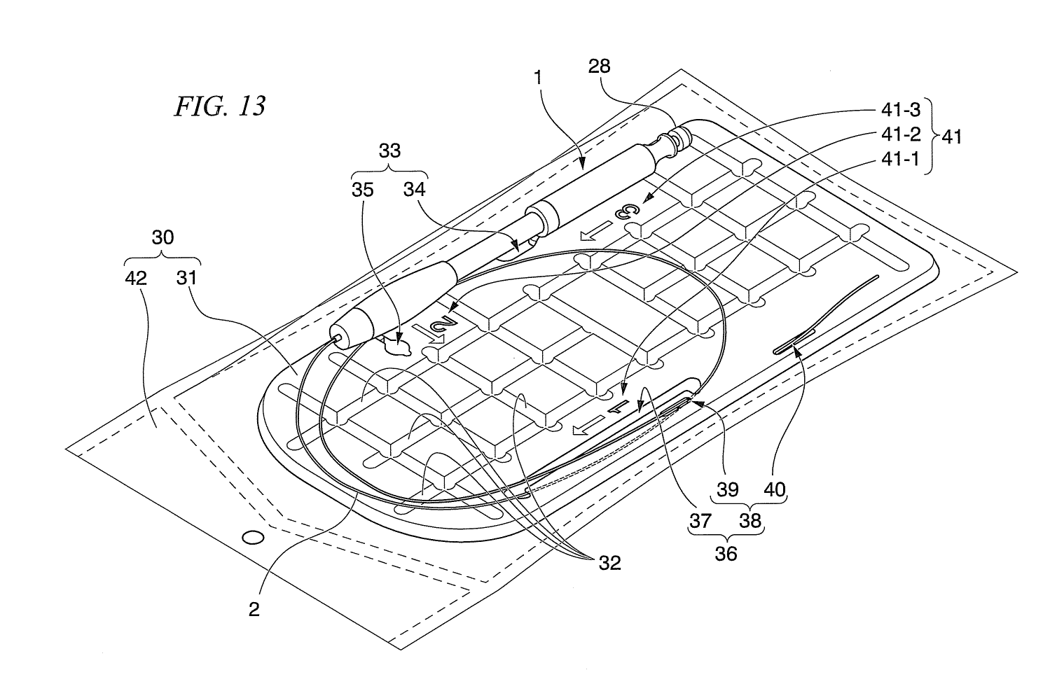

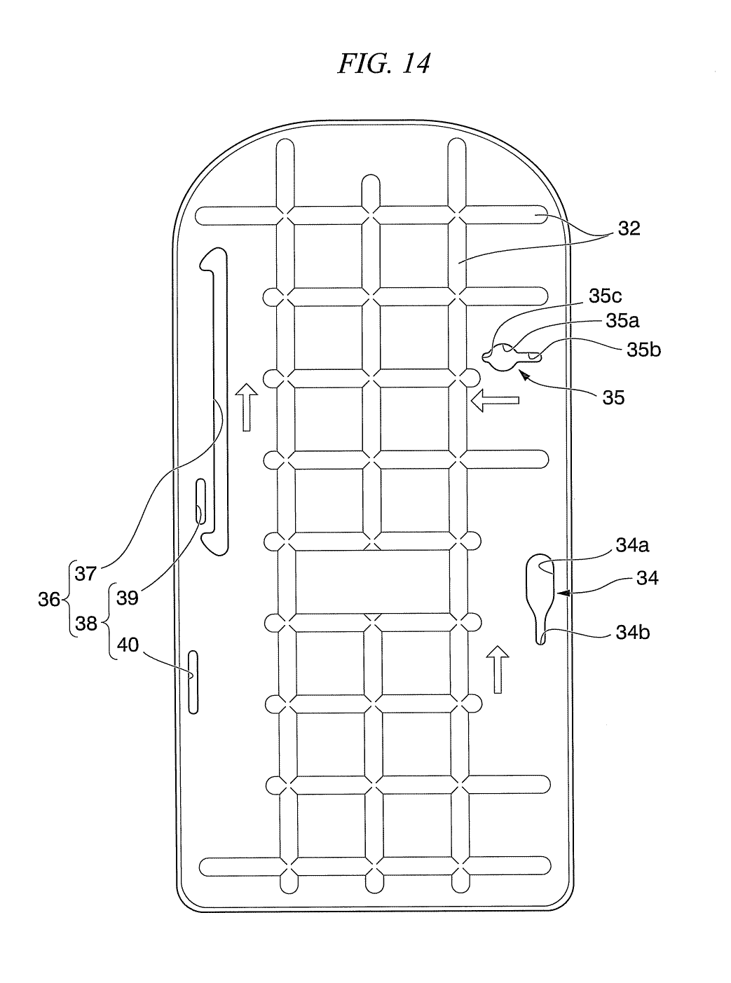

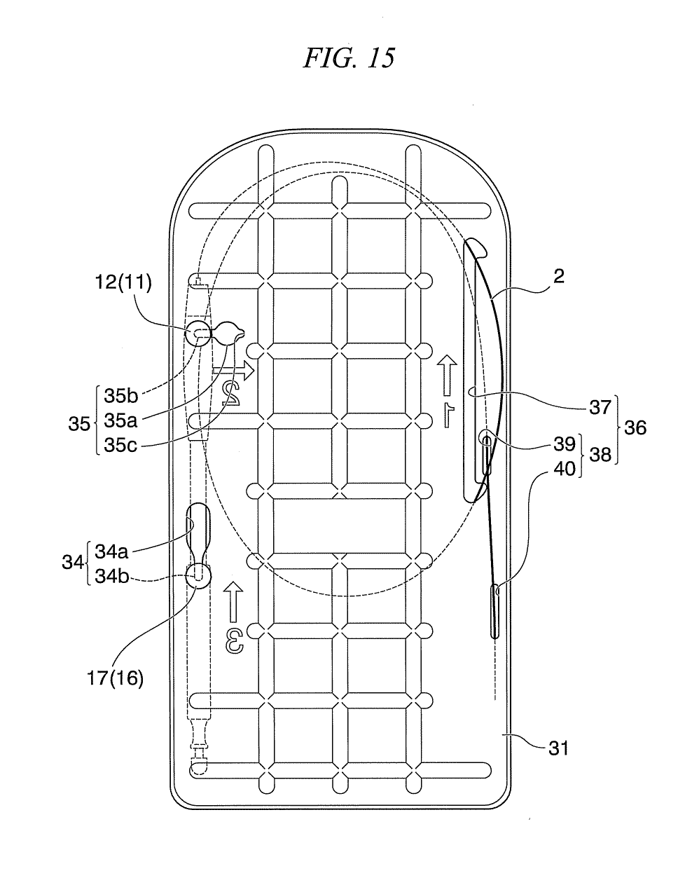

Next, the configuration of the storage bag 30 that is used when shipping and storing the treatment instrument 1 will be explained with reference to FIGS. 13 to 15. FIG. 13 is a perspective view showing the arrangement in which the treatment instrument 1 is stored inside the storage bag 30. FIG. 14 is a planar view of the tray 31 in the storage bag 30. FIG. 15 is a rear view showing the arrangement in which the treatment instrument 1 is attached to the tray 31.

In general, treatment instruments from medical use are stored in a sterile state prior to use. When there is a long inserted part, such as the inserted body 2 of the treatment instrument 1 in this embodiment, it has been the conventional practice to wind the inserted body into a looped state and store it in this form inside a bag or the like.

However, because the inserted body 2 in the present embodiment tends to rebound to a linear form due to the elasticity of the stylette 27 and the needle barrel 3, the sterile state of the inserted body 2 may be broken through contact with a person, another instrument, the floor, etc., due to incorrect opening technique when removing the inserted body 2.

In order to resolve this problem, the treatment instrument 1 of this embodiment is provided stored in a special storage bag 30.

As shown in FIG. 13, the storage bag 30 is provided with a tray 31 in which the treatment instrument 1 is attached, and a sack-like cover 42 within which tray 31 with the attached treatment instrument 1 is stored.

As shown in FIG. 13 and FIG. 14, the tray 31 is formed to be approximately rectangular in shape, and has a reinforced part 32 consisting of convexities and concavities extending in a lattice-like form. In addition, a screw engaging part 33 (first screw engaging part 34, second screw engaging part 35), an inserted body engaging part 36 for engaging the inserted body 2, and a marking 41 which displays the sequence for removing the treatment instrument 1 from the tray 31 during use of the treatment instrument, are formed to the tray 31.

As shown in FIG. 14, the first screw engaging part 34 and the second screw engaging part 35 are provided to one side of the two long sides of the tray 31. The inserted body engaging part 36 is provided to the other side of the two long sides of the tray 31.

As shown in FIG. 15, the first screw engaging part 34 is formed by the edge of a through hole which is formed passing through the width direction of the tray 31 for the purpose of attaching the screw 16, which is attached to the slider stopper 14 of the treatment instrument 1, to the tray 31. The first screw engaging part 34 has a screw insertion part 34a, into which the large diameter part 17 of the screw 16 can be inserted, and a shaft inserted part 34b into which the shaft of the screw 16 can be inserted. Shaft inserted part 34b is narrower in width than the large diameter part 17 of the screw 16 and is connected at one end to the screw insertion part 34a.

In this embodiment, the screw insertion part 34a and the shaft insertion part 34b are formed in the shape of a long oval which is directed from the distal end to the proximal end of the operation section 8 when the treatment instrument 1 is attached in the tray 31.

The second screw engaging part 35 is formed by the edge of a through hole which is formed passing through the width direction of the tray 31 for the purpose of attaching the screw 11, which is attached to the grip 10 of the treatment instrument 1, to the tray 31. The second screw engaging part 35 has a screw insertion part 35a into which the large diameter part 12 of the screw 11 can be inserted; a shaft insertion part 35b into which the shaft of the screw 11 can be inserted, and which is connected at one end to the screw insertion part 35a and which has a width that is narrower than the large diameter part 12 of the screw 11; and a withdrawal stop part 35c formed on the opposite side from the shaft insertion part 35b, so that with the screw insertion part 35a is between the withdrawal stop part 35c and the shaft insertion part 35b.

In this embodiment, the second screw engaging part 35 extends in a direction perpendicular to the direction in which the first screw engaging part 34 extends.

As shown in FIGS. 14 and 15, the inserted body engaging part 36 has an engaging piece 37 that extends in the inward direction of the tray 31 when viewed from the planar perspective of the tray 31, and a distal end engaging part 38 which engages with the distal end of the inserted body 2.

The part of the inserted body 2 that is bent in a loop is engaged by the engaging piece 37. In addition, as shown in FIG. 13, the portion of the looped inserted body 2 that is opposite the engaging piece 37 is held by the outer surface of the operation section 8 which is attached to the first screw engaging part 34 and the second screw engaging part 35. As a result, the inserted body 2 is held stably between the engaging piece 37 and the operation section 8.

Further, as shown in FIG. 15, due to pushing pressure from the looped inserted body 2 on the operation section 8, the distal end part of the operation section 8 is pushed from the screw insertion part 35a toward the shaft insertion part 35b. As a result, when the inserted body 2 is engaged with the inserted body engaging part 36, the screw 11 cannot readily come loose from the second screw engaging part 35.

Two through holes 39,40 into which the inserted body 2 can be inserted are formed to the distal end engaging part 38. When the distal end of the inserted body 2 is pushed into the through holes at the distal end engaging part 38 with the inserted body 2 held between the engaging piece 37 and the operation section 8, the distal end of the inserted body 2 is pushed against the edge of the through holes 39,40 of the distal end engaging part 38 due to the force acting to return the looped inserted body 2 to the linear state. As a result, the distal end of the inserted body 2 does not easily pull out from the through holes 39,40 at the distal end engaging part 38.

As shown in FIG. 13, the marking 41 uses numbers to display the sequence of steps for releasing the treatment instrument 1 from the tray 31, and is formed when the tray 31 itself is formed. The marking 41 has a first marking 41-1 in which the number [1] is displayed near the engaging piece 37, a second marking 41-2 in which the number [2] is displayed near the second screw engaging part 35, and a third marking 41-3 in which the number [3] is displayed near the first screw engaging part 34. Arrows showing the direction for releasing the various parts of the treatment instrument 1 from the tray 31 are provided to the first marking 41-1, the second marking 41-2, and the third marking 41-3, respectively.

The marking 41 is provided for the purpose of prompting the user to remove the various parts of the treatment instrument 1 from the tray 31 in order from number [1] to number [3]. By releasing the treatment instrument 1 in sequence from number [1] to number [3] from the tray 31, it is possible to prevent the inserted body 2 from unintentionally returning to the linear form and causing the inserted body 2 to contact a non-sterile surface such as the human body, floor, wall, etc. The specific method employed when removing the treatment instrument 1 from the tray 31 will be explained below.

As shown in FIG. 13, the cover 42 is made of a material that has a high permeability with respect to the sterilizing gas and which does not transmit bacteria. In this embodiment, the cover 42 is formed to have a space for holding the treatment instrument 1 by layering a roughly rectangular shaped piece of antibacterial paper and a permeable resin film, and then fixing these in place by heat-sealing the periphery.

The storage bag 30 is designed so that the cover 42 is heat fused with the treatment instrument 1 attached to the tray 31 and the treatment instrument 1 held inside the space in which bacteria are prevented from entering. After the cover 42 has been closed by heat sealing, the storage bag 30 is sterilized using a sterilizing gas such as ethylene oxide gas or by irradiating with an electron beam or gamma rays.

A method for using a treatment instrument 1 designed as described above and the actions during such operation will now be explained.

When using treatment instrument 1, the treatment instrument 1 is housed inside the storage bag 30 as shown in FIG. 13 and is removed from the storage bag 30 which is supplied in a sterilized state. Note that the treatment instrument 1 is provided in an arrangement in which the stylette 27 is inserted into the needle barrel 3 and the knob 28 of the stylette 27 is screwed into the proximal end of the needle slider 23.

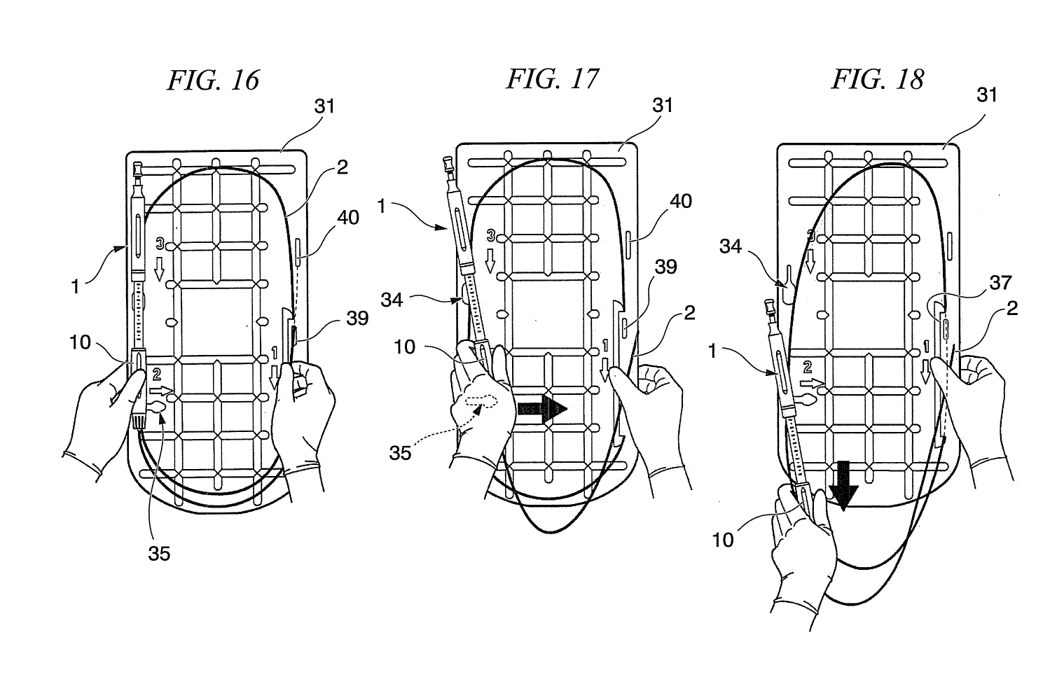

Specifically, the cover 42 is first torn and the tray 31 in which the treatment instrument 1 is attached is removed from inside the cover 42. Next, as shown in FIG. 16, the inserted body 2 which is in the area labeled with the number [1] in the tray 31 is gripped, and the distal end of the inserted body 2 is pulled out from the through hole 40 which is formed in the tray 31.

After pulling out the distal end of the inserted body 2 from the through hole 40, the user holds the distal end of the inserted body 2 together with the tray 31 as shown in FIG. 17, and holds the grip 10 with the hand opposite that holding the inserted body 2. Next, the user moves the large diameter part 12 of the screw 11 which is in the area labeled with the number [2] in the tray 31 toward the engaging piece 37 side, and releases the large diameter part 12 from the second screw engaging part 35.

After releasing the large diameter part 12 from the second screw engaging part 35, the user releases the part of the inserted body 2 that is engaged in the engaging piece 37 from the engaging piece 37 while holding the distal end of the inserted part 2 as shown in FIG. 18. Next, the user holds both the grip 10 and the inserted body 2 that is positioned near the grip 10, and releases the large diameter part 17 of the screw 16 that is provided to the operation section 8 from the first screw engaging part 34 which is in the area labeled with the number [3] in the tray 31.

The treatment instrument 1 is thus released from the tray 31 in the order of numbers [1], [2], [3] as per the markings 41 formed to the tray 31. As a result, it is possible to easily release the treatment instrument 1 from the tray 31 without releasing the loop of the inserted body 2 and having the distal end thereof accidentally contact a non-sterile area.

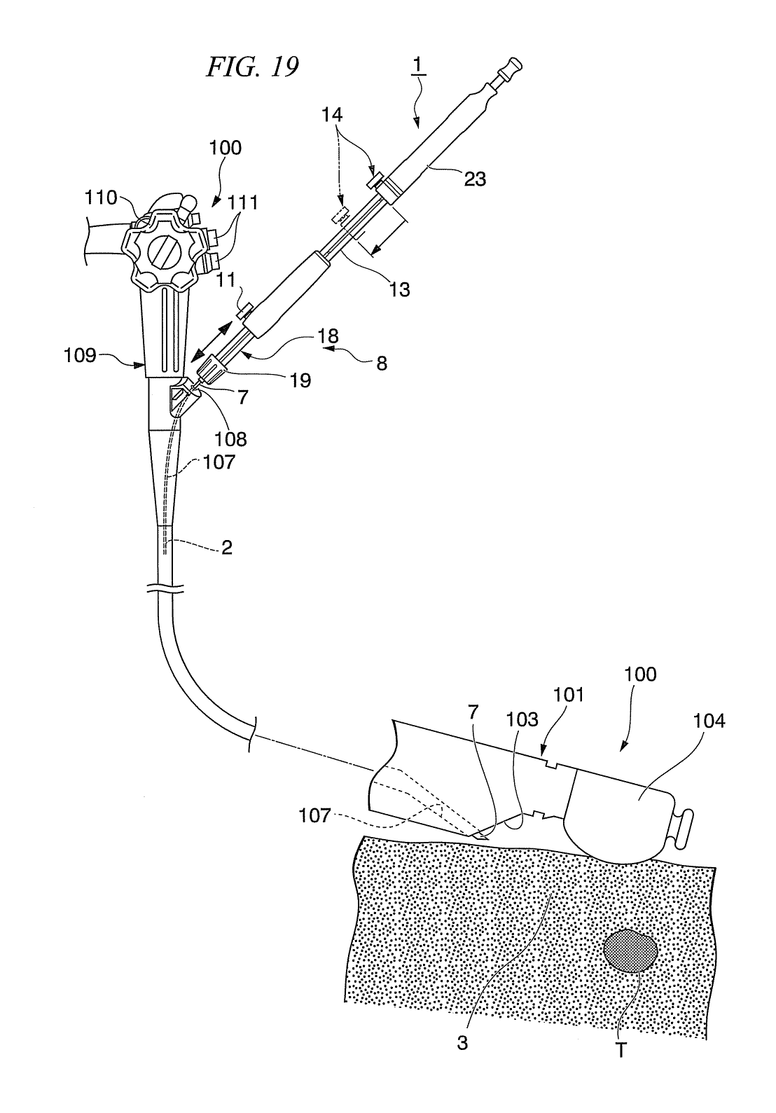

Next, a procedure will be performed using the treatment instrument 1 together which the ultrasonic endoscope 100 shown in FIG. 1. FIGS. 19 through 22 are views for explaining the action when using the treatment instrument 1.

This embodiment explains as an example a biopsy procedure in which the needle barrel 3 of the treatment instrument 1 is pierced into tissue in which a pathological change is present within a deep part of the tissue inside the body, and cells from the diseased area are collected through the inside of the needle barrel 3, as shown in FIG. 19.

As shown in FIG. 19, the user introduces the inserted part 101 of the ultrasonic endoscope 100 into the body, and employs an optical image capture mechanism 103 to optically visualize the area where the procedure is to be performed. In addition, it is also possible to use the ultrasonic scanning mechanism 104 to visualize a deep area located where procedure is to be performed.

Next, the area where the biopsy is to be performed is determined based on the results of observation using the optical image capture mechanism 103 and the ultrasonic scanning mechanism 104.

Next, the user inserts the distal end side of the inserted body 2 of the treatment instrument 1 from the distal end mouthpiece 108, provided to the operation section 109 of the ultrasonic endoscope 100, into the channel 107. The user then fixes the fixing screw 19, which is provided to the operation section 8 of the treatment instrument 1, to the distal end mouthpiece 108. As a result, the treatment instrument 1 is fixed in place to the ultrasonic endoscope 100.

Next, the user loosens the screw 11 which is provided to the grip 10, and, while observing the sheath 7 and the inside of the body using the optical image capture mechanism 103, suitably adjusts the amount that the sheath 7 projects out from the distal end of the inserted part 101 of the ultrasonic endoscope 100 using the sheath adjuster 18.

Next, based on the results of observation using the ultrasonic scanning mechanism 104, the user moves the slider stopper 14 to align with the position of the target tissue T where the biopsy is to be performed, and fixes the slider stopper 14 to the slide rail 13. By doing so, the maximum length that the needle barrel 3 can be projected out from the sheath 7 is restricted to the length at which the needle slider 23 comes into contact with the slider stopper 14.

Next, as shown in FIG. 20, the user pushes the needle slider 23 toward the distal end side of the operation section 8. As a result, as shown in FIG. 21, the needle barrel 3 projects out from the sheath 7. Next, as shown in FIG. 22, the distal end of the needle barrel 3 pierces the tissue and is advanced by pushing into the target tissue T where the biopsy is to be performed.

The needle barrel 3 that is externally exposed from the surface of the tissue can be observed using the optical image capture mechanism 103, and the lateral part of the distal end of the needle barrel 3 which is pushed into the tissue can be observed using the ultrasonic scanning mechanism 104.

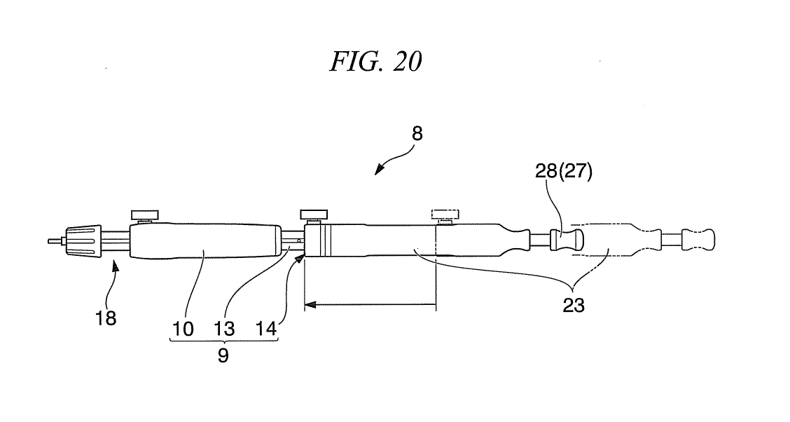

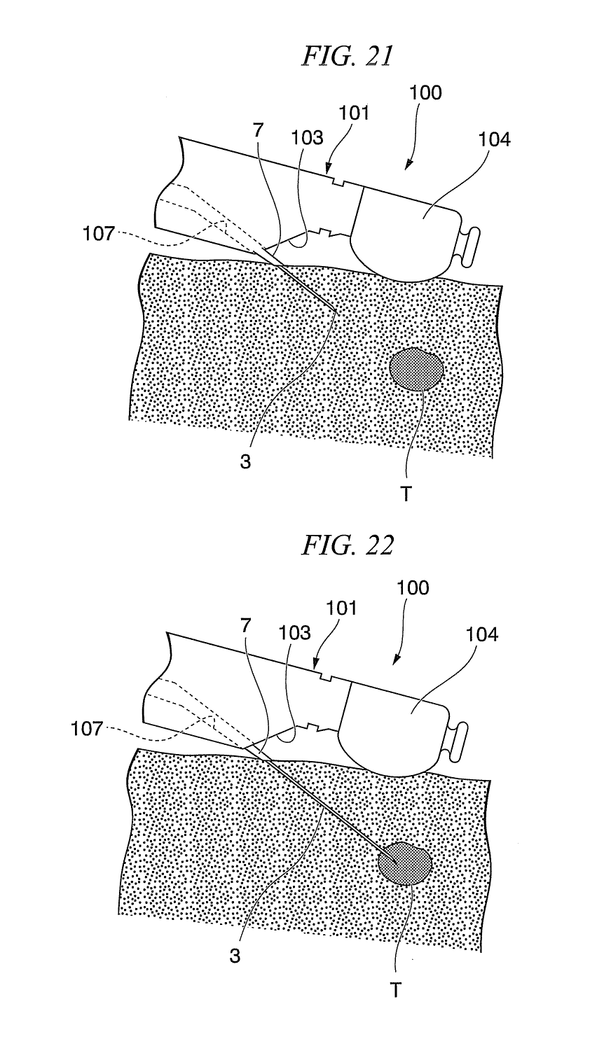

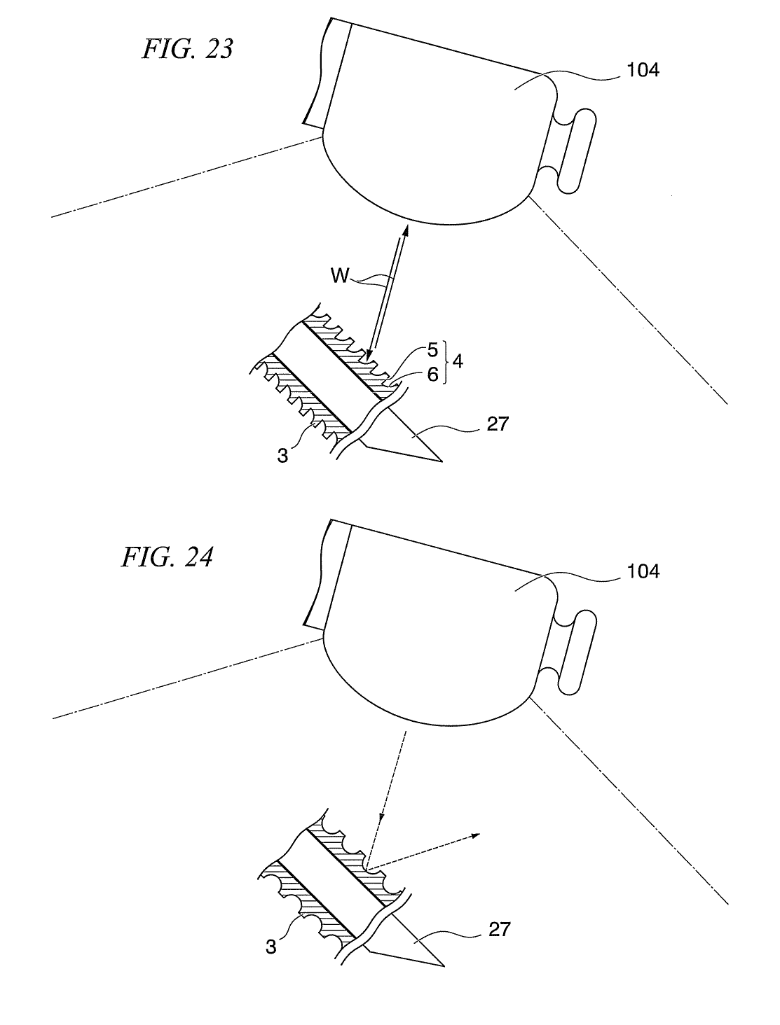

FIG. 23 is a schematic view showing the reflected state of the ultrasound waves when the ultrasonic scanning mechanism 104 is operated when the needle barrel 3 has punctured the tissue. FIG. 24 is a schematic view showing the reflected state of the ultrasound waves in a needle barrel having the conventional dimples.

The ultrasonic scanning mechanism 104 is provided with an ultrasonic vibrating element for generating ultrasound waves and an ultrasonic receiver for receiving the ultrasound waves. As shown in FIG. 23, in this embodiment, the ultrasonic scanning mechanism 104 scans the biological tissue and the needle barrel 3 within the ultrasound scanning ranges that is directed toward the needle barrel 3 which is projecting out from the sheath 7.

Ultrasound waves W radiated by the ultrasonic vibrating element are reflected by the outer surface of the needle barrel 3. The concave surface part 6 of each of the dimples 4 at the distal end of the needle barrel 3 forms a curved surface, so that at least a portion of the concave surface part 6 is able to reflect the ultrasound waves W toward the ultrasonic receiver.

Next, the conventional dimples will be explained as an example for comparative purposes. When a simple hemispherical dimple is formed to the needle barrel as shown in FIG. 24 for example, one quarter of the hemisphere on the proximal end side of the needle barrel disperses the ultrasound wave W beyond the ultrasound scanning limits.

As shown in FIG. 23 and FIG. 24, in contrast to the simple hemispherical dimples, in this embodiment, a planar surface part 5 is substituted for the surface that cause dispersion of the ultrasound waves W beyond the ultrasound scanning limits. As a result, the concave surface part 6 which focuses the ultrasound waves W within the ultrasound scanning limits is disposed at higher density than in the conventional dimples. As a result, it is possible to focus more ultrasound waves W within the ultrasound wave scanning limits of the ultrasonic scanning mechanism 104 as compared to the simple hemispherical dimples (see FIG. 24). Thus, the needle barrel 3 can be reflected more clearly on the ultrasound screen.

The user is able to employ the ultrasonic observation section 115 shown in FIG. 1 to visualize the ultrasound image based on the ultrasound waves received at the ultrasonic scanning mechanism 104. The user references the image of the needle barrel 3 that is reflected clearly on the ultrasonic observation section 115, and moves the distal end of the needle barrel 3 to the target tissue T where the biopsy is to be performed. Note that at the stage where the distal end of the needle barrel 3 arrives at the target tissue T to be biopsied, the tissue does not enter inside the needle barrel 3 because the stylette 27 is inserted in the needle barrel 3.

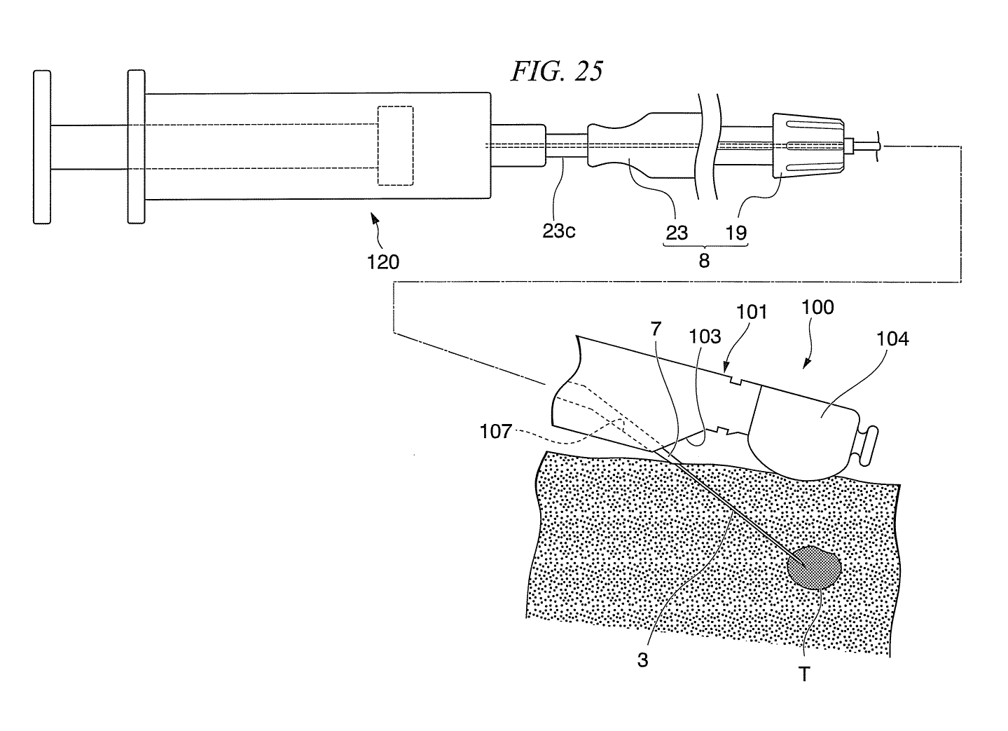

Next, the user releases the knob 28 from the needle slider 23 by turning the knob 28 of the stylette 27 shown in FIG. 2. The stylette 27 is then pulled out from the operation section 8 and the inserted body 2. As a result, a through hole is formed which extends from the distal end of the needle barrel 3 to the proximal end of the needle slider 23 as shown in FIG. 25. The user then fixes a suitable syringe 120 to the screw thread 23c that is formed to the proximal end of the needle slider 23, this syringe 120 having a distal end engages with the screw thread 23c. Next, the inside of the needle barrel 3 is suctioned using the syringe 120, and the cells, etc. from the target tissue T which is to be biopsied are suctioned into the syringe 120 from the distal end of the needle barrel 3.

Once a required amount of cells, etc. have been suctioned into the syringe 120, the needle slider 23 is pulled all the way toward the proximal end side of the operation section 8 and the distal end of the needle barrel 3 is housed inside the sheath 7. As a result, the needle barrel 3 is withdrawn from the tissue. Once the needle barrel 3 is pulled out from the tissue, the fixing screw 19 is released from the distal end mouthpiece 108 of the operation section 109 of the ultrasonic endoscope 100, and the treatment instrument 1 is withdrawn from the channel 107.

This concludes the serial medical treatment.

The use of ultrasonic endoscopy to perform a medical procedure while obtaining an image of a needle barrel is conventionally known. For example, in the example of a treatment instrument disclosed in Japanese Patent Application, Publication No: 2003-190179, a plurality of annular grooves are formed to the needle barrel, and the treatment instrument is used by projecting the distal end of the needle barrel out from the distal end of the channel of the ultrasonic endoscope. In this example, the image of the needle barrel on the ultrasound image is formed by reflection at the annular grooves of the ultrasound waves radiated from the proximal end side toward the distal end side of the needle barrel. More specifically, only the surface which is positioned at the distal end side of the needle barrel and directed toward the proximal end side of the various annular grooves formed in the outer surface of the needle barrel contributes to the reflection of the ultrasound waves toward the ultrasonic receiver side.

The present inventors has noted that a useless area which does not contribute to the reflection of the ultrasound waves toward the ultrasonic receiver side is present in the conventional treatment instrument when the treatment instrument is employed together with an ultrasonic endoscope. Moreover, the present inventors discovered that if the useless area is reduced in size and the areas that contribute to the reflection of the ultrasound waves toward the ultrasonic receiver side are disposed at high density, then it is possible to increase the ultrasound waves that are reflected toward the ultrasonic receiver side.

In the treatment instrument 1 according to the present embodiment, for each of the various dimples 4 respectively, the length component that is measured along the center axis of the needle barrel 3 and is the distance between the deepest part 4c of the dimple 4 and the rear edge 4b of dimple 4 is formed to be shorter than the length component that is measured along the center axis and is the distance between the deepest part 4c and the front edge of dimple 4. As a result, it is possible to form more surfaces to the outer surface of the needle barrel 3 that can reflect the ultrasound waves toward the ultrasonic receiver when ultrasound waves are radiated from the proximal end side toward the distal end side of the needle barrel 3. Accordingly, the number of reflected waves that are reflected from the outer surface of the needle barrel 3 toward the ultrasonic receiver is increased, making the image of the needle barrel 3 in the ultrasound image clearer.

In addition, the various dimples 4 that are formed to the outer surface of the needle barrel 3 have a concave surface part 6 at the distal end side thereof. Accordingly, there is little increase or decrease in reflected waves even if the position of the needle barrel 3 is moved with respect to the ultrasonic scanning mechanism 104. As a result, the image of the needle barrel 3 can be reflected in the ultrasound image with roughly constant brightness even if the needle barrel 3 is moved.

Note that when employing the treatment instrument 1 according to the present embodiment together with the ultrasonic endoscope 100, there are few cases where observation of the needle barrel 3 is carried out by radiating ultrasound waves from the distal end to the proximal end side of the needle barrel 3; rather, the observation is carried out by radiating the ultrasound waves from the proximal end to the distal end side of the needle barrel 3. In the treatment instrument 1 according to the present embodiment, the dimples 4 are provided which are particularly suitably capable of reflecting ultrasound waves when the ultrasound waves are radiated from the proximal end side to the distal end side of the needle barrel 3. Thus, a particularly superior effect can be obtained when using the treatment instrument 1 together with the ultrasonic endoscope 100.

### Example Modification 1

Next, a modification of the treatment instrument 1 explained in the preceding embodiment will be explained below.

Note that in the case of this modification and the Example Modification 2 below, components having the same structure as those of the treatment instrument described above will be labeled with the same numeric symbol and a redundant explanation thereof will be omitted here.

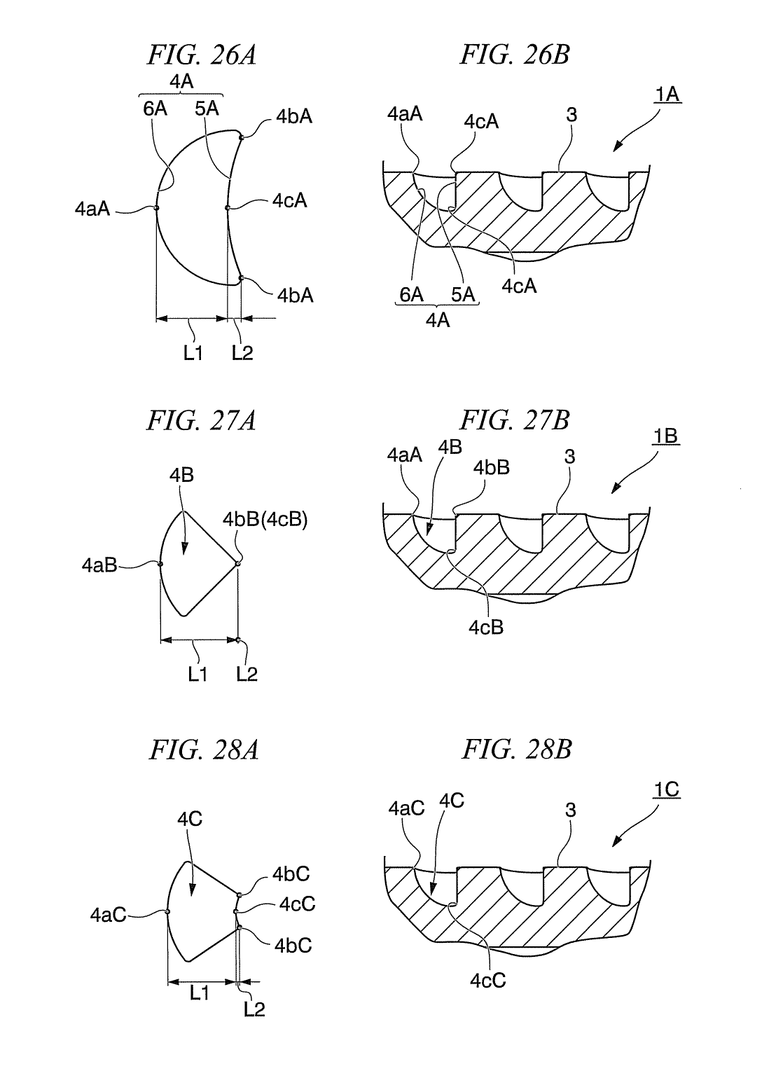

FIG. 26A and FIG. 26B are schematic views for explaining the shape of the dimples 4A in this modification.

As shown in FIG. 26A and FIG. 26B, the treatment instrument 1A of this modification differs in structure from the above-described treatment instrument 1 in that dimples 4A which have a differently shaped inner surface are provided in place of the dimples 4 to the needle barrel 3.

The inner surface of a dimple 4A has a curved surface part 5A, which forms a portion of a circular cylindrical surface employing a line perpendicular to the center axis of the needle barrel 3 as its center of rotation and comprises a convexity that is directed at the distal end of the needle barrel 3, and a concave surface part 6A, which consists of a concave surface formed at a position that includes the front edge 4a of the dimple 4.

The front edge 4aA of each of the dimples 4A is positioned closer to the distal end side than the positions on the curved surface that are closest to the distal end. The concave surface part 6A forms part of the spherical surface.

For each of the various dimples 4A, the length component L2 which is measured along the center axis of the needle barrel 3 and is the distance between the rear edge 4bA positioned closest to the proximal end of the needle barrel 3 and the deepest part 4cA of the dimple 4A is shorter than the length component L1 which is measured along the center axis and is the distance between the front edge 4aA positioned closest to the distal end and the deepest part 4cA.

As in the case of the treatment instrument 1 explained in the preceding embodiments, when ultrasound waves are radiated from the proximal end side to the distal end side of the needle barrel 3, the concave surface part 6A can be employed to reflect the ultrasound waves toward the ultrasonic receiver in this modification as well.

### Modification 2

Next, another modification of the treatment instrument 1 explained in the preceding embodiment will be explained below.

FIG. 27A and FIG. 27B are schematic views for explaining the shape of the dimples 4B in this modification.

As shown in FIG. 27A and FIG. 27B, the treatment instrument 1B of this modification differs in structure from the previously described treatment instrument 1 in that dimples 4B having a differently shaped inner surface are provided in place of the dimples 4 to the needle barrel 3.

The inner surface of the dimples 4B is shaped to form part of the outer surface of one-eighth of a spherical body created when a sphere is sectioned by three planes which mutually intersect passing through the sphere.

For each of the various dimples 4B, the length component L2, which is measured along the center axis of the needle barrel 3 and is the distance between the rear edge 4bB, which is positioned closest to the proximal end of the needle barrel 3, and the deepest part 4cB of the dimple 4B, is substantially zero, and is shorter than the length component L1 which is measured along the center axis and is the distance between the front edge 4aB, which is positioned closest to the distal end, and the deepest part 4cB.

In this embodiment, it is possible to form more dimples 4B to the outer surface of the needle barrel 3 than the aforementioned dimples 4 or dimples 4A. As a result, it is possible to further increase the number of surfaces capable of reflecting the ultrasound waves toward the ultrasonic receiver.

### Modification 3

Next, another modification of the treatment instrument 1 explained in the preceding embodiment will be explained below.

FIG. 28A and FIG. 28B are schematic views for explaining the shape of the dimples 4C in this modification.

As shown in FIG. 28A and FIG. 28B, the treatment instrument 1C of this modification differs in structure from the previously described treatment instrument 1 in that dimples 4C having a differently shaped inner surface are provided in place of the dimples 4 to the needle barrel 3.

The inner surfaces of the dimples 4C are shaped to have an arch-like outline which forms a convexity on the distal end side of the needle barrel 3 and which are mutually concentric on the respective distal end side and proximal end side of the needle barrel 3.

For each of the various dimples 4C, the length component L2, which is measured along the center axis of the needle barrel 3 and is the distance between the rear edge 4bC, which is positioned closest to the proximal end of the needle barrel 3, and the deepest part 4cC of the dimple 4C, is shorter than the length component L1 which is measured along the center axis and is the distance between the front edge 4aC positioned closest to the distal end and the deepest part 4cC.

Even by forming dimples 4C of this shape to the needle barrel 3, it is possible to further increase the surfaces capable of reflection of the ultrasound waves toward the ultrasonic receiver.

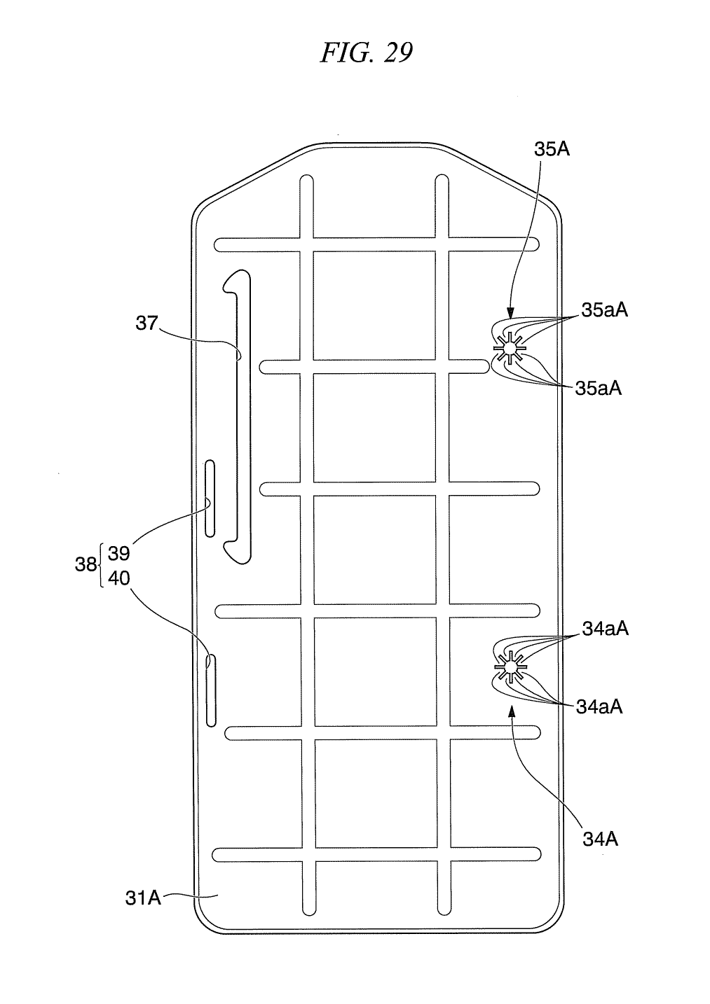

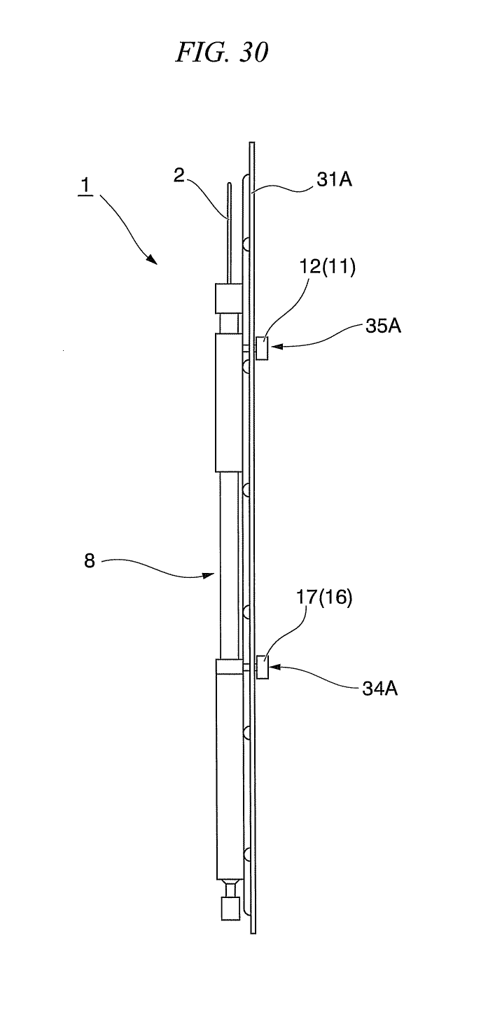

Next, an example of a modification of the storage bag explained in the preceding embodiment will be explained with reference to FIG. 29 and FIG. 30.

FIG. 29 is a planar view showing the tray in the storage bag in this modification. FIG. 30 is a side view showing the arrangement in which the treatment instrument is attached to the tray in this modification.

This modification differs with respect to the provision of a tray 31A in place of the tray 31.

As shown in FIG. 29 and FIG. 30, a first screw engaging part 34A is formed in place of the first screw engaging part 34, and a second screw engaging part 35A is formed in place of the second screw engaging part 35 in this tray 31A.

The first screw engaging part 34A has a plurality of flaps 34aA which can be spread apart by pushing with the head of the screw. The distal end of the respective flaps 34aA is formed to have an approximately circular outline which is slightly larger than the diameter of the shaft of the screw 16. The large diameter part 17 of the screw 16 is inserted into the first screw engaging part 34A, and the outer peripheral surface of the screw 16 is held by the distal end of the respective flaps 34aA.

A flap 35aA which is equivalent in shape and size to the first screw engaging part 34A is formed to the second screw engaging part 35A.

When releasing the treatment instrument 1 from the tray 31A in this modification, the treatment instrument 1 is pulled in a direction which is perpendicular to the surface on which the first screw engaging part 34A and the second screw engaging part 35A are formed. As a result, the respective flaps 34aA,35aA are spread apart by the screw 11 and the screw 16, and each of the screws are released from the first screw engaging part 34A and the second screw engaging part 35A.

As in the case of the embodiment explained above, it is possible to remove the treatment instrument 1 from the tray 31A without the inserted body 2 coming into contact with a non-sterile surface in this modification as well.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

For example, the above-described dimples 4, dimples 4A, dimples 4B or dimples 4C may be in mutual contact along the peripheral direction of the needle barrel 3. As a result, it is possible to form the dimples 4, dimples 4A, dimples 4B or dimples 4C at even higher density to the outer surface of the needle barrel 3.

The preceding embodiments employed a needle barrel 3 as an example of a treatment instrument, however, the treatment instrument is not limited thereto. For example, it is also acceptable to employ a solid needle as the treatment device and to form the dimples 4 to the outer surface of the needle. In addition, a probe or the like which is inserted into the biologic tissue and generates radio waves deep within the tissue may be cited as another example of a treatment instrument.

Note that it is also acceptable to suitably combine the structural elements shown in the preceding embodiments and modifications.

The present invention is not limited to the preceding explanation, but only by the appended claims.

What is claimed is:

1. A treatment instrument that is inserted in a freely advancing and retracting manner into the channel of an ultrasonic endoscope, the treatment instrument comprising:
a treatment member that has a distal end and a proximal end and an outer surface that is a circular cylindrical shape in which a plurality of dimples are formed indenting into the outer surface, wherein
the length component that is measured in the direction of the center axis of the treatment member and which is the distance between the rear edge, which is positioned closest to the proximal end, and the deepest part of the dimple, is shorter than the length component that is measured in the direction of the center axis and which is the distance between the front edge which is closest to the distal end and the deepest part.

2. A treatment instrument according to claim 1, wherein the inner surface of the dimple has a planar surface part which is parallel to the plane that intersects with the center axis and a concave surface part consisting of a concave surface that is formed closer to the distal end side than the planar surface part.

3. A treatment instrument according to claim 1, wherein the inner surface of the dimple is provided with a curved surface that forms a portion of the circular cylindrical surface having as its rotational center of the straight line intersecting with the center axis, and which consists of a convexity directed at the distal end, wherein the front edge is positioned closer to the distal end side than the location along the curved surface that is positioned closest to the distal end.

4. A treatment instrument according to claim 2 or 3, wherein the concave surface part forms a part of a spherical surface.

5. A treatment instrument according to claim 1 wherein the inner surface of the dimple is shaped to form part of the outer surface of one-eighth of a spherical body created when a sphere is sectioned by three planes which mutually intersect passing through the sphere.

6. A treatment instrument according to one of claims 1 through 5, wherein the dimple is formed in rows around a circumferential direction of an outer surface of the treatment member.

7. A treatment instrument according to claim 6 wherein the dimples are in mutual contact about the circumferential direction.
